(19) United States
(12) Patent Application Publication
(10) Pub. No.: US 2012/0189694 A1
(43) Pub. Date: Jul. 26, 2012

(54) CO-PRECIPITATE COMPRISING A PHOSPHODIESTERASE-5 INHIBITOR (PDE-5-INHIBITOR) AND A PHARMACEUTICALLY COMPATIBLE CARRIER, PRODUCTION AND USE THEREOF

(75) Inventors: Katrin Rimkus, Muenchen (DE); Frank Muskulus, Groebenzell (DE)

(73) Assignee: ratiopharm GmbH, Ulm (DE)

(21) Appl. No.: 13/387,276

(22) PCT Filed: Jul. 8, 2010
(86) PCT No.: PCT/EP10/04181

§ 371 Date: Apr. 10, 2012

(30) Foreign Application Priority Data
Jul. 29, 2009 (DE) 10 2009 035 211.2

(51) Int. Cl. A61K 47/32; A61K 47/36; A61K 47/26; A61K 31/496; A61K 31/53; A61K 9/00; A61K 31/4985; A61K 9/20; A61P 13/00; A61P 19/10; A61P 9/10; A61P 9/12; A61P 29/00; A61P 35/00; A61P 3/10; A61P 13/10; B02C 23/08; A61K 47/38

(52) U.S. Cl. 424/465; 514/772.6; 514/781; 514/778; 514/777; 514/252.16; 514/243; 424/400; 514/250; 424/464; 241/24.1

(57) ABSTRACT

The invention relates to a co-precipitate comprising a phosphodiesterase-5 inhibitor (PDE-5-inhibitor) and a pharmaceutically compatible copolymer carrier comprising 2 or more different acrylic acid derivatives, a method for production thereof and a medication comprising the co-precipitate according to the invention, a method for producing said medication and the use of said medication for treating an illness wherein the inhibiting of phosphodiesterase-5 is of therapeutic benefit.

The present invention relates to a coprecipitate comprising a phosphodiesterase 5 inhibitor (PDE5 inhibitor) and a pharmaceutically acceptable copolymer carrier consisting of 2 or more different acrylic acid derivatives, to processes for production thereof and to a medicament comprising the inventive coprecipitate, to processes for producing this medicament and to the use of this medicament for treatment of a disorder in which the inhibition of phosphodiesterase 5 is of therapeutic benefit.

Numerous active ingredients of potential interest for use as medicaments entail the disadvantage that they are only sparingly soluble in aqueous solution or in water. Associated with the sparing solubility of these active ingredients is an only slow release of the active ingredient. There is thus no guarantee of rapid bioavailability, which is thus sufficient for the effect to be achieved, of the active ingredient in the body.

Especially phosphodiesterase 5 inhibitors (PDE5 inhibitors), for example sildenafil, vardenafil or tadalafil, feature the disadvantage of sparing solubility in aqueous solutions or in water. This impairs both the processing and the bioavailability thereof.

One means of improving the solubility of sparingly soluble active ingredients is based on increasing the surface area of the active ingredient particles by grinding or micronization, as disclosed in WO 01/08688. WO 01/08688 discloses oral formulations with rapid release. The desired solubility or release has been achieved by comminuting the tadalafil particle size to below 40 μm. However, the grinding or micronization of active ingredients can entail disadvantages, for example the formation of agglomerates. This gives rise to poorly definable particle sizes which in turn have poorly definable solubility. Moreover, any possible static charging of the active ingredient has an adverse effect on processability. A further possible disadvantage is the poor flowability of the ground active ingredient. Particularly pressing to tablets or filling of capsules require further processing steps, for example granulation. In spite of small particles, it is often necessary to add a large amount of surfactant to achieve sufficient solubility. Finally, the production thereof is complicated and inconvenient.

A process for producing a solids dispersion comprising a sparingly soluble active ingredient is described in WO 96/38131. The solubility of the active ingredient is said to be improved by coprecipitation, but attempts to release the tadalafil active ingredient have shown that tablets containing these coprecipitates release the active ingredient very slowly (see also example 1 of the present document).

It was therefore an object of the present invention to provide coprecipitates with a phosphodiesterase 5 inhibitor (PDE5 inhibitor) of sparing solubility in aqueous solution, which release the active ingredient faster than the coprecipitates known from the prior art and thus ensure rapid bioavailability of the phosphodiesterase 5 inhibitor (PDE5 inhibitor) in the body.

It has now been found that, surprisingly, a coprecipitate comprising a phosphodiesterase 5 inhibitor (PDE5 inhibitor) of sparing solubility in aqueous solution or in water and at least one pharmaceutically acceptable carrier, wherein the pharmaceutically acceptable carrier is a copolymer consisting of 2 or more different acrylic acid derivatives, and/or the pharmaceutically acceptable carrier is a cellulose acetate, a starch derivative or an oligosaccharide, ensures rapid release and bioavailability of the phosphodiesterase 5 inhibitor (PDE5 inhibitor).

The term “bioavailability” is used here as known to those skilled in the art and refers to a pharmaceutical parameter for the proportion of a substance which is available unchanged in systematic circulation. The bioavailability indicates how rapidly and to what extent the substance is absorbed and is available at the site of action.

Precipitation is the term for the process of fully or partly precipitating a dissolved substance, by addition of suitable substances, as an insoluble precipitate in the form of for example, crystals, flakes or droplets. In this process it is unimportant whether the precipitant alters the chemical composition of the dissolved substance. In a specific form of precipitation, called coprecipitation, the precipitate of a substance is mixed with another substance which is present in the solvent, and which is incorporated into the precipitate in the course of precipitation.

A “precipitate” is understood in the general sense to mean a precipitate of a chemical compound in the presence of substances which are soluble. A coprecipitate is accordingly a precipitate of a chemical compound mixed with another substance.

M. A. Khan et al. (S.T. Pharma Sciences 7 (6) 483-490, 1997) report coprecipitates of ibuprofen with acrylic ester and methacrylic ester polymers, such as Eudragit and Carbopol. Tablets produced from ibuprofen/Eudragit S100 coprecipitates, however, do not release the active ingredient virtually completely within a very short period, but instead have a controlled release of the active ingredient over a period of 8 hours. It was thus all the more surprising that the rapid release (and not a controlled release over a long period) required in the case of phosphodiesterase 5 inhibitors (PDE5 inhibitors), which have become famous particularly as impotence drugs (e.g. Viagra), was observed with the inventive coprecipitates.

The present invention therefore provides a coprecipitate comprising a phosphodiesterase 5 inhibitor (PDE5 inhibitor) and at least one pharmaceutically acceptable carrier, wherein the pharmaceutically acceptable carrier is a copolymer consisting of 2 or more different acrylic acid derivatives of the general formula (I)

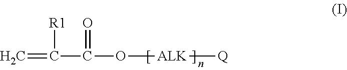

where, in any of the 2 or more different acrylic acid derivatives, each independently,
R1 is H or a straight-chain or branched C1-C6 alkyl radical,

is 0 or 1,

ALK is a straight-chain or branched C1-C6 alkyl radical,
Q is H or —OR2, —NR2R3 or —N⁺R2R3R4, where R2, R3 and R4 are each independently a straight-chain or branched C1-C6 alkyl radical,

and/or the pharmaceutically acceptable carrier is a cellulose acetate, a starch derivative or an oligosaccharide.

Phosphodiesterases (PDEs), to be exact 3′,5′-cyclonucleotide phosphodiesterases, are a group of enzymes which degrade second messengers such as cAMP and cGMP to AMP and GMP. Due to their involvement in the signal transduction of cells, they are a target of pharmacological interest. They are divided into seven subtypes which have different locations in the tissues in the human organism. Phosphodiesterase 5 (PDE5) is the name of one of the enzymes which cleave the phosphoric ester bond in cGMP to form 5′-GMP. In humans, phosphodiesterase 5 occurs in the smooth muscle of the penile cavernous body (corpus cavernosum penis and the pulmonary artery. Blockage of cGMP degradation by inhibition of PDE5 (for example with sildenafil) leads to increased signals of the relaxation signaling pathways, and specifically to increased blood supply in the corpus cavernosum penis, and to lowering of pressure in the blood vessels of the lung.

The term “phosphodiesterase 5 inhibitor (PDE5 inhibitor)” therefore refers generally to compounds which inhibit PDE5 by specific interaction, i.e., for example, not by denaturation or the like. This increases the cGMP concentration, which leads, for example, to relaxation of the muscles and hence to an erection in the penis, or to a drop in blood pressure in the lung. The uses of PDE5 inhibitors include treatment of erectile dysfunction and of pulmonary arterial hypertension.

More particularly, specific inhibition may be understood to mean inhibition where the respective PDE5 inhibitor inhibits PDE5 with an IC₅₀ of less than 100 nM, especially of less than 10 nM.

In this context, the terminus IC₅₀ is a measure of the efficacy of a compound in inhibiting a particular phosphodiesterase enzyme (PDE enzyme), in the present case PDE5. The IC₅₀ indicates the concentration of a compound which leads to 50% inhibition of the enzyme in a single dose response experiment. The IC₅₀ for a compound can be determined, for example, by a known in vitro method, as described in general terms in Y. Cheng et al., Biochem. Pharmacol., 22, pp. 3099-3108 (1973). Preferred PDE5 inhibitors are selective for the inhibition of PDE5, i.e. they inhibit PDE5 preferentially over other phosphodiesterases. In addition, such PDE5 inhibitors are characterized by the following characteristics:
(1) an IC₅₀ for the inhibition of PDE5 at least 100 times lower than the IC₅₀ for the inhibition of PDE6;
(2) an IC₅₀ for the inhibition of PDE5 at least 1000 times smaller than the IC₅₀ for the inhibition of PDE1c; and
(3) an IC₅₀ of less than 10 nM for the inhibition of PDE5.

Preferred PDE5 inhibitors inhibit PDE5 selectively compared to PDE6 and PDE1c. This selectivity is reflected by the differences in the IC₅₀. This difference is expressed as the PDE6/PDE5 ratio of IC₅₀ values, i.e. the ratio of the IC₅₀ for the inhibition of PDE6 to the IC₅₀ for the inhibition of PDE5 (PDE6/PDE5) is greater than 100, preferably greater than 300 and more preferably greater than 500.

Similarly, the ratio of the IC₅₀ for the inhibition of PDE1c to the IC₅₀ for the inhibition of PDE5 (PDE1c/PDE5) is greater than 1000. Preferred inhibitors exhibit a more than 3000-fold difference between the inhibition of PDE5 and PDE1c, preferably a more than 5000-fold difference between the IC₅₀ values for the inhibition of PDE5 and PDE1c. The efficacy of the inhibitor, as represented by the IC₅₀ for the inhibition of PDE5, is less than 10 nM, preferably less than 5 nM, more preferably less than 2 nM and most preferably less than 1 nM.

Examples of usable PDE5 inhibitors include the following substances: zaprinast, MY5445, dipyridamol, vardenafil, sildenafil and tadalafil. Further PDE5 inhibitors are described, for example, in U.S. Pat. No. 6,548,490; US 2003/0139384, WO 94/28902 and WO 96/16644. The properties of inventive coprecipitates are advantageous especially in the case of sparingly soluble PDE5 inhibitors, and so particular preference is given to coprecipitates which comprise sparingly soluble PDE5 inhibitors, especially those which are sparingly soluble in water or aqueous solutions.

A “carrier” is generally understood to mean a substance with which other substances are associated, and into which, according to the present matter, substances can also be intercalated. Thus, such a substance can “carry” another substance. In the widest sense, the term “carrier” encompasses one or more solid or liquid carriers compatible with one another and with the active pharmaceutical constituent. The carriers in the present invention are preferably solid.

The term “pharmaceutically acceptable” relates to a substance which generally does not cause any significant irritation, complications of any kind or even toxicity in the subject treated, and does not reduce, or even raises, the biological activity and properties of the active constituent, or interacts therewith.

The term “polymer” relates to a chemical compound which consists of chain or branched molecules (macromolecules) formed from identical or equivalent units, called the monomers. In connection with the present invention, the term “copolymers” consequently relates to polymers composed of two or more different monomer units.

The term “acrylic acid” in connection with the present invention is used as known to the person skilled in the art, and relates to propenoic acid, also known as 2-propenoic acid, ethylenecarboxylic acid or vinylcarboxylic acid.

The term “acrylic acid derivative” used in connection with the term “copolymer” relates to acrylic acid and derivatives thereof, this relating both to the esters of acrylic acid and to the derivatives of methacrylic acid, for example butyl methacrylate, 2-dimethylaminoethyl methacrylate or methyl methacrylate. These acrylic acid derivatives can be used to prepare copolymers suitable as carriers.

Suitable copolymers suitable as carriers include the acrylic polymers of the Eudragit brands (Evonik Röhm GmbH, Darmstadt). One example in this connection is Eudragit E, which is a copolymer with cationic character based on dimethylaminoethyl methacrylate and uncharged methacrylic esters in a ratio of 1:2:1 with a mean molar mass of approx. 150 000, the chemical name of which is poly[butyl methacrylate, 2-dimethylaminoethyl methacrylate, methyl methacrylate].

Further Eudragit brands include Eudragit FS 30 D (copolymer of methacrylic acid, methacrylate and methyl methacrylate in a ratio of 10:65:25), Eudragit L brands (copolymers based on methacrylic acid and methyl methacrylate or ethyl acrylate), Eudragit NE 30 D (copolymer of uncharged character based on ethyl acrylate and methyl methacrylate), Eudragit RL brands (copolymers based on acrylic and methacrylic esters with a low content of quaternary ammonium groups), Eudragit RS brands (copolymers based on acrylic and methacrylic esters) and Eudragit S brands (copolymers based on methacrylic acid and methyl methacrylate).

Preference is given in the context of the present invention to Eudragit E with the chemical name poly[butyl methacrylate, 2-dimethylaminoethyl methacrylate, methyl methacrylate].

The cellulose (empirical formula (C₆H₁₀O₅)ₙ) is the main constituent of plant cell walls (proportion by mass 50%) and is thus the world's most common organic compound. Cellulose is therefore also the most common polysaccharide. It is an unbranched polysaccharide consisting of several hundreds to tens of thousands of β-D-glucose molecules ((1→4)-glycosidic bond) or cellobiose units. Cellulose is formed in the plasma membrane and crosslinks internally to form fibrilar structures. The spatial arrangement of the cellulose fibrils is controlled by the microtubuli. In industry, cellulose is obtained in the form of pulp from wood.

The term “cellulose acetate” in connection with the present invention is used as known to those skilled in the art and relates to acetic esters of cellulose, which can be prepared industrially by reaction of linters or pulp with acetic anhydride in acetic acid or methylene chloride as a solvent using strong acids such as sulfuric or perchloric acid as catalysts in a batchwise process. Cellulose acetate is also referred to colloquially as acetyl cellulose; the original trade name thereof is Lonarit. Cellulose acetate is a thermoplastic polymer which has varying transparency and is suitable as a tablet binder.

A preferred cellulose acetate in the context of the present invention is cellulose acetate phthalate (also known by the abbreviation C-A-P among others), which can be prepared by reaction of a partial acetate ester of cellulose with phthalic anhydride. A further preferred cellulose acetate in the context of the present invention is hydroxypropylmethylcellulose acetate phthalate.

Starch is a polysaccharide which has the formula (C₆H₁₀O₅)ₙ and consists of α-D-glucose units joined to one another via glycosidic bonds. The macromolecule therefore belongs to the carbohydrates. It is normally present in the plant cell in the form of organized grains. These starch grains are of different size and shape according to the plant type. Starch consists of variable percentages of each of amylose (approx. 20-30%), linear chains with helical (screw) structure with only α-1,4-glycosidic linkages, and of amylopectin (approx. 70-80%) with α-1,6-glycosidie and α-1,4-glycosidic linkages.

Derivatizations of starch can be performed to pursue different aims, some of which include achievement of lowering of the gelatinization temperature thereof, increasing the solution stability thereof, or influencing other (solution) properties via the variation of the polar character of the polysaccharides. Modifications may, for example, via a change in the amylose/amylopectin ratio, pregelatinization, partial hydrolytic degradation or chemical derivatization of the starches.

Examples of starch derivatives in connection with the inventive coprecipitate are starch, starch esters such as xanthogenates (O,S-dialkyl esters), and corresponding acetates, phosphates, sulfates and nitrates, and crosslinked starches or substituted n-octenylsuccinate of starch.

The term “oligosaccharides” is used here as known to those skilled in the art and relates to molecules which result from condensation of 2 to about 10 monosaccharides. This can form linear, branched and cyclic oligosaccharides. In contrast to the polysaccharides, the properties of the oligosaccharides still correspond substantially to those of the monosaccharides. Oligosaccharides occur in free form principally in the world of plants and consist predominantly of hexoses, and less commonly of pentoses or amino sugars. Disaccharides, trisaccharides and tetrasaccharides are used.

Preferred oligosaccharides in the context of the present invention are disaccharides. The term “disaccharides” is used as known to those skilled in the art and relates to carbohydrates, which usually have the empirical formula C₁₂H₂₂O₁₁ and are formed from two monosaccharide molecules linked by a glycosidic bond (D-glucose, D-fructose, inter alia). Disaccharides occur in free form, such as sucrose, as constituents of oligo- and polysaccharides (cellobiose), or are glycosidically bonded to plant dyes including constituents (to aglycones such as anthocyanidines). The most important disaccharides are cellobiose, maltose (malt sugar), lactose (milk sugar) and sucrose (cane sugar). Preferred disaccharides in connection with the inventive coprecipitate arc maltose, lactose and sucrose.

Moreover, the cellulose acetate in the inventive coprecipitate is cellulose diacetate, cellulose triacetate, an incomplete hydrolysate thereof, cellulose acetate phthalate or cellulose acetate butyrate, particular preference being given to cellulose acetate butyrate or cellulose acetate phthalate. In addition, the starch derivative in the inventive coprecipitate is a crosslinked starch, an acetylated starch or a substituted n-octenylsuccinate of starch.

In a preferred embodiment, the present invention relates, in one embodiment, to a coprecipitate, wherein the copolymer of 2 or more, especially 2, 3, 4 or 5, particularly 3, different acrylic acid derivatives of the general formula (I), where, in each of the 2 or more different acrylic acid derivatives, each independently,
R1 is H or a straight-chain C1-C4 alkyl radical, methyl, ethyl, propyl or butyl, especially methyl,
n is 0 or 1, especially 1,
Alk is a straight-chain C1-C4 alkylene radical, methylene, ethylene, propylene or butylene, especially methylene, ethylene or butylene,
Q is H or —NR2R3 where R2 and R3 are each independently a straight-chain C1-C4 alkyl radical, methyl, ethyl, propyl or butyl, especially methyl.

Alternatively or additionally, the cellulose acetate is cellulose diacetate, cellulose triacetate, an incomplete hydrolysate thereof or cellulose acetate butyrate, especially cellulose acetate butyrate or cellulose acetate phthalate, and/or the starch derivative is a crosslinked starch, an acetylated starch or a substituted n-octenylsuccinate of starch, and/or the oligosaccharide is a disaccharide such as maltose, lactose or sucrose.

Especially preferably, the copolymer in the inventive coprecipitate is a Eudragit of the E brand, the chemical name of which is poly[butyl methacrylate, 2-dimethylaminoethyl methacrylate, methyl methacrylate].

The term “solution” is used here as known to those skilled in the art and relates in the widest sense to homogeneous mixtures of different substances, where even the tiniest volume components have an equivalent composition. Solutions in the narrower sense are understood to mean liquid mixtures of at least two components in which the partners are present in molecular dispersion in different ratios. In a solution, at least one component has the function of a solvent; therefore, also conceivable are solutions in which two solvents are used for dissolution of the substance, preferably of a solid substance. The particles of the dissolved substance in a solution are surrounded by a solvate shell of the solvent(s).

A solution in connection with the inventive coprecipitate is also a solution which, as well as salts, may additionally also comprise, for example, acids or bases, preferably acids.

A preferred solvent here is water. An aqueous solution is accordingly a solution comprising predominantly water as a solvent.

The solubility of a substance states the extent to which a pure substance can be dissolved in a solvent. It thus refers to the property of a substance of mixing with the solvent with homogeneous distribution (as atoms, molecules or ions). The solvent is usually a liquid.

The liquids in which a solid has good solubility depend on the molecular properties of the substance and of the liquid. Thus, salt-type substances (ionic compounds) are soluble almost only in polar solvents such as water or else hydrogen fluoride (HF). Many lipophilic substances, for example of the wax type, in contrast, have significant solubility only in organic solvents such as benzine (an “apolar” solvent). “Polar” in this context means that the molecules of the solvent have a dipole moment and therefore interact with charged molecules (ions), or molecules which are themselves polar, of the substance to be dissolved, but without any reaction. The polarity of solvents is scalable. Different polarities and hence different solubilities are used extensively in chromatography processes.

Classification of the solubilities is possible via the maximum amount of substance dissolved. Solubilities below 0.1 mg/ml of dissolved substance are referred to as sparing solubility, between 10 and 33 mg/ml as moderate solubility, and greater than 100 mg/ml as good solubility. One example of a sparingly soluble substance is tadalafil, which according to WO 01/08687 has solubility of only about 2 μg/ml in water.

In one embodiment of the present invention, the phosphodiesterase 5 inhibitor (PDE5 inhibitor) is of sparing solubility in water. Accordingly, the present invention preferably relates to a coprecipitate comprising a phosphodiesterase 5 inhibitor (PDE5 inhibitor) and a pharmaceutically acceptable carrier, wherein the phosphodiesterase 5 inhibitor (PDE5 inhibitor) is of sparing solubility in water.

The classification of a substance can also relate to other pure solvents. For instance, the term “sparingly soluble in water” relates to the above-defined sparing solubility of a substance in water as a pure solvent.

In a preferred embodiment, the present invention relates to a coprecipitate comprising a phosphodiesterase 5 inhibitor (PDE5 inhibitor) and a pharmaceutically acceptable constituent, wherein the phosphodiesterase 5 inhibitor (PDE5 inhibitor) is sparingly soluble in an aqueous solution, especially in water, and is sildenafil, vardenafil or tadalafil, especially tadalafil.

As well as sildenafil (Viagra®), which is probably the best known PDE5 inhibitor to the wider public, tadalafil in particular has also been found to be an extremely effective PDE5 inhibitor, Tadalafil (IUPAC name: (6R,12aR)-6-(1,3-benzodioxol-5-yl)-5-methyl-1,2,3,6,7,12,12a-octahydropyrazino[2,1:6,1]pyrido[3,4-b]indole-1,4-dione) has been used, for example, in the form of oral formulations for treatment of erectile dysfunction (see, for example, WO 01/08688). This active ingredient can be prepared, for example, according to Doughan A. et al. (2003), J. Med. Chem., 46, 4533-4542, in which it is referred to as (6R,12aR)-2,3,6,7,12,12a-hexahydro-2-methyl-6-(3,4-methylenedioxyphenyl)pyrazino[2′,1′:6,1]pyrido[3,4-b]indole-1,4-dione.

As mentioned above, PDE5 cleaves the phosphoric ester bond in cGMP to form 5′-GMP. As well as PDE5, there are further, exclusively cGMP-cleaving phosphodiesterases. However, they differ functionally from PDE5, for example in that they require cofactors or also cleave CAMP. If they are involved in the transmission of visual signals, they are designated with the number 6, and, if they require manganese as a cofactor, with the number 9. PDE11 cleaves both cAMP and cGMP.

Preferred PDE5 inhibitors which are used in the context of the present invention are sildenafil, vardenafil and tadalafil. Sildenafil is the international non-proprietary name for the compound 1-{[3-(1-methyl-7-oxo-3-propyl-6,7-dihydro-1H-pyrazolo[4,3-d]pyrimidin-5-yl)-4-ethoxyphenyl]sulfonyl}-4-methylpiperazine, which is better known by the trade name Viagra® and is marketed for treatment of erectile dysfunction in men. Sildenafil was the first drug from the active ingredient class of the PDE5 inhibitors. Vardenafil is the non-proprietary name of the active ingredient 1-{[3-(5-methyl-4-oxo-7-propyl-3,1-dihydroimidazo[5,1-f][1,2,4]triazin-2-yl)-4-ethoxyphenyl]sulfonyl}-4-ethylpiperazine, which is marketed under the Levitra® or Vivanza® trade name for treatment of erectile dysfunction. Tadalafil (IUPAC name (6R,12aR)-6-(1,3-benzodioxol-5-yl)-2-methyl-1,2,3,4,6,7,12,12a-octahydropyrazino[2′,1′:6,1]pyrido[3,4-b]indole-1,4-dione) is likewise known for treatment of erectile dysfunction under the Cialis® trade name.

With a half-life of 17.5 hours, tadalafil, however, has a much greater half-life compared to sildenafil and vardenafil. While the action lasts for 4 to 6 hours in the case of sildenafil and for 8 to 12 hours in the case of vardenafil, it can last up to 36 hours in the case of tadalafil. Usually, the effect sets in one hour after administration. In the context of the present invention, tadalafil is therefore a preferred PDE5 inhibitor.

The present invention therefore relates, in a further embodiment, to a coprecipitate as defined above, comprising a phosphodiesterase 5 inhibitor (PDE5 inhibitor) and a pharmaceutically acceptable carrier, wherein the phosphodiesterase 5 inhibitor (PDE5 inhibitor) is sildenafil, vardenafil or tadalafil, especially tadalafil.

In the inventive coprecipitate, the phosphodiesterase 5 inhibitor (PDE5 inhibitor) and the pharmaceutically acceptable carrier are present in a weight ratio of 1:2 to 2:1, for example in a weight ratio of 1:2, 1:1 and 2:1, although weight ratios with odd-numbered proportions are also possible in principle. Preferably, in the inventive coprecipitate, the phosphodiesterase 5 inhibitor (PDE5 inhibitor) and the pharmaceutically acceptable carrier are present in a weight ratio of 1:1.

Accordingly, the present invention relates, in a further preferred embodiment, also to a coprecipitate comprising a phosphodiesterase 5 inhibitor (PDE5 inhibitor) and a pharmaceutically acceptable carrier, wherein the phosphodiesterase 5 inhibitor (PDE5 inhibitor) and the pharmaceutically acceptable carrier are each as defined above, and wherein the phosphodiesterase 5 inhibitor (PDE5 inhibitor) and the pharmaceutically acceptable carrier are present in a weight ratio of 1:2 to 2:1, preferably in a weight ratio of 1:1.

The term “weight ratio” is understood to mean a statement of the ratio formed by the masses of at least two substances or components used relative to one another. In contrast to the simple statement of the exact mass of the substances in question used in each case, the weight ratio merely states the constant ratio of the masses of substances used. Thus, the mass ratio is a generally valid statement regarding the masses of at least two substances to be used, and is therefore not restricted to a specific example, in contrast to the exact statement of masses.

In a further embodiment, in the inventive coprecipitate, the phosphodiesterase 5 inhibitor (PDE5 inhibitor) is enclosed by the pharmaceutically acceptable carrier.

The term “enclosed” in connection with the inventive coprecipitate means that the phosphodiesterase 5 inhibitor (PDE5 inhibitor) is enveloped by the pharmaceutically acceptable carrier in such a manner that the phosphodiesterase 5 inhibitor (PDE5 inhibitor) is substantially enclosed by the carrier.

The term “enclosed” in connection with the inventive coprecipitate also includes a coprecipitate in which the phosphodiesterase 5 inhibitor (PDE5 inhibitor) is completely enclosed by the pharmaceutically acceptable carrier, but is in no way restricted to such a case. Therefore, the term “enclosed” in the context of the present invention also refers to those coprecipitates in which the phosphodiesterase 5 inhibitor (PDE5 inhibitor) is not enclosed completely by the pharmaceutically acceptable carrier, and accordingly also coprecipitates in which the phosphodiesterase 5 inhibitor (PDE5 inhibitor) is enclosed only partly by the pharmaceutically acceptable carrier.

In particularly visual examples, the phosphodiesterase 5 inhibitor (PDE5 inhibitor) may be enclosed in the manner of a mesh, ribbon or spiral by the pharmaceutically acceptable carrier, in which cases there is only inadequate, if any, overlapping of the respective regions of the carrier to form a complete shell around the phosphodiesterase 5 inhibitor (PDE5 inhibitor). In the simplest case, the phosphodiesterase 5 inhibitor (PDE5 inhibitor) is enclosed by the pharmaceutically acceptable carrier in such a way that the envelope that the pharmaceutically acceptable carrier forms around the phosphodiesterase 5 inhibitor (PDE5 inhibitor) is not complete but has “holes”. Preferably, the phosphodiesterase 5 inhibitor (PDE5 inhibitor) is completely enclosed by the pharmaceutically acceptable constituent.

The present invention further provides a process for producing the inventive coprecipitate, comprising the steps of:
a) dissolving the phosphodiesterase 5 inhibitor (PDE5 inhibitor) and the pharmaceutically acceptable carrier in a mixture of an aprotic polar solvent and a protic solvent;
b) coprecipitating the phosphodiesterase 5 inhibitor (PDE5 inhibitor) and the pharmaceutically acceptable carrier by increasing the protic character of the mixture of the solvents; and
c) removing the coprecipitate from the mixture of the solvents.

The terms “coprecipitate”, “phosphodiesterase 5 inhibitor (PDE5 inhibitor)” and “pharmaceutically acceptable carrier” are used here as defined above.

In principle, a coprecipitate is produced from the precipitation of at least two substances, one substance in this case enveloping the other substance, preferably completely. In the present case, the phosphodiesterase 5 inhibitor (PDE5 inhibitor) is that substance which is enclosed by the auxiliary.

The term “dissolution” is very well-known to the person skilled in the art and refers in the context of the present invention to the transfer of at least one solid substance into the liquid phase in a solvent. In this connection, it is likewise known to those skilled in the art that parameters such as selection of the temperature of the mixture of the solvents and of the polarity of each individual solvent, and stirring of the solution, can influence the dissolving operation.

The term “solvent” is used here as known to those skilled in the art and refers generally to substances which can cause other substances to dissolve by a physical route, in the narrower sense inorganic and organic liquids which are capable of dissolving other gaseous, liquid or solid substances. A prerequisite for suitability as a solvent is that neither the dissolving nor the dissolved substance undergoes significant chemical change in the dissolving operation, i.e. that the components of the solution can be recovered in the original form by physical separation processes such as distillation, crystallization, sublimation, vaporization or absorption. In this connection, it is known to those skilled in the art that the process of dissolution of solid substances in a solvent can be improved by deprotonation or protonation of the at least one substance to be dissolved by the solvent. The protonation or deprotonation of the at least one substance to be dissolved by the solvent is therefore not a chemical reaction of this substance. In a wider sense—particularly in industry—the term “solvent” is frequently also understood to mean mere dispersants which are liquid under standard temperature and pressure conditions and serve to dissolve, to emulsify or to suspend other substances, in order to enable the processing (e.g. paint thinners) or else removal (e.g. stain removers) thereof.

Among the inorganic solvents are firstly the proton-containing (or hydrogen-containing) solvents, for example H₂O, liquid NH₃, H₂S, hydrogen cyanide and HNO₃, and proton-free solvents (liquid SO₂, N₂O₄, NOCl, SeOCl₂, ICl, BrF₃, AsCl₃, HgBr₂ etc.), and secondly the aqueous and nonaqueous solvents.

The group of nonaqueous solvents also includes the organic solvents. Representatives of the organic solvents are the alcohols, for example methanol, ethanol, propanols, butanols, octanols, cyclohexanol, glycols (ethylene glycol, diethylene glycol), ethers and glycol ethers (diethyl ether, dibutyl ether, anisole, dioxane, tetrahydrofuran, mono-, di-, tri-, polyethylene glycol ether), ketones (acetone, butanone, cyclohexanone), esters (acetic esters, glycol esters), amides and other nitrogen compounds (dimethylformamide, pyridine, N-methylpyrrolidone, acetonitrile), sulfur compounds (carbon disulfide, dimethyl sulfoxide, sulfolane), nitro compounds (nitrobenzene), halohydrocarbons (dichloromethane, chloroform, tetrachloromethane, tri-, tetrachloromethane, 1,2-dichloroethane, chlorofluorocarbons), hydrocarbons (benzine, petroleum ether, cyclohexane, methylcyclohexane, decalin, terpene solvents, benzene, toluene, xylenes).

The term “protic solvent” refers to those solvents which contain or release protons and/or can form hydrogen bonds, for example water, alcohols, amines, etc. The release of protons from a molecule of a solvent is also referred to as dissociation. The most important protic solvent is water, which dissociates (expressed in simplified terms) to a proton and a hydroxide ion. Further protic solvents are, for example, alcohols, in the ease of which the proton is always released on the hydroxyl group, since the electronegative oxygen can readily accept the negative charge which forms. In the borderline case, carboxylic acids may also be protic solvents, provided that (in simplified terms) release of protons from the carboxylic acid does not cause a chemical change in the substance to be dissolved. A further group of protic solvents is that of the amines, which firstly contain protons in their amino group, and also can accept a proton in each case by virtue of the free electron pair on the nitrogen atom of the amino group.

As already mentioned above, one characteristic feature of protic solvents is that they can form hydrogen bonds. The term “hydrogen bond” is used here as known to those skilled in the art, and refers to a bond which forms between a hydrogen atom covalently bonded to an atom of an electronegative element (proton donor, X) and the free electron pair of another electronegative atom (protonacceptor, Y). In general, such a system is formulated as RX—H—Y—R′, the dotted bond symbolizing the hydrogen bond. Possible X are mainly O (oxygen), N (nitrogen), S (sulfur) and halogens; in some eases (e.g. HCN), C (carbon) can also function as a proton donor. The polarity of the covalent bond of the proton donor causes a positive partial charge of the hydrogen atom, while the proton acceptor, being an atom of an electronegative element, bears a corresponding negative partial charge. Y in turn is an atom selected differently than. X from the main possible elements O (oxygen), N (nitrogen), S (sulfur) and halogens. For example, a hydrogen bond can be formed between the hydrogen atoms of water (hydrogen donor) and the free electron pair of the nitrogen in amines (hydrogen acceptor). It should also be noted that particular functional groups can act simultaneously as a hydrogen donor and as a hydrogen acceptor. A simple example of this is that of hydroxyl groups, or the hydrogen bonds between water molecules.

The formation of the above-described hydrogen bonds not only influences the solubility characteristics of protic solvents, but also the further properties thereof. Thus, given comparable molecular size, for example, the respective boiling point of protic solvents is generally well above those of aprotic solvents.

In contrast to the protic solvents discussed above, what are called aprotic solvent molecules do not possess a functional group from which hydrogen atoms can be released in the form of protons. Consequently, such aprotic solvents thus do not dissociate.

In addition, a distinction is made between what are called aprotic polar solvents and what are called aprotic apolar solvents. Nonpolar substances are composed of nonpolar molecules Which themselves do not have a permanent electrical dipole moment.

An illustrative aprotic apolar solvent is an alkane, in which all the hydrogen atoms are bonded equally firmly to the carbon atoms. Therefore, protons can dissociate off only with very great difficulty to form carbanions, which are very reactive themselves. The substances of such pure hydrocarbons are therefore very readily soluble in one another, but are not dissolved by polar substances such as esters, and by protic substances such as water. In the liquid, the particles are held together merely by van der Waals forces (temporary dipoles due to the fluctuation in electron density distribution). Therefore, in this substance group, the boiling temperatures compared to molecular size and mass are much lower than in the case of permanent dipoles.

Aprotic polar substances are discussed after the polar substances.

Polar substances are understood in the widest sense to mean those substances which consist of polar molecules which in turn feature a permanent electric dipole moment.

The term “polarity” refers in chemistry to formation of separate charge centers resulting from movement of charges in atom groups, the effect of which is that an atom group is no longer electrically neutral. The electrical dipole moment therefore also serves as a measure of the polarity of a molecule.

Polar compounds are firstly those with an ionic bond (polar or heteropolar bond) and secondly those with an electrical dipole moment and polarized covalent bond. Cyclohexanol is referred to, for example, as a polar solvent, while cyclohexane is a nonpolar solvent.

The polarity of an overall molecule is caused by a polar atomic bond, or in the extreme case by ionic bonding. Polar bonds are notable for inhomogeneous distribution of bonding electrons between the bonding partners. If atoms of different electronegativity are bonded, the result is such a polarization of the bond. If only polarized atomic bonds are present in a molecule, the individual dipole moments of the bonds add up vectorially to give an overall dipole moment. If this overall dipole moment has the magnitude of zero, the substance is nevertheless nonpolar, for example CO₂ or tetrachloromethane. If, however, there is a permanent non-zero overall dipole moment, the molecule is polar, for example water. According to the size of this overall dipole moment, a substance is more or less polar. The transition is therefore fluid from extremely polar to completely nonpolar. On the basis of their polarity, solvents are ordered in an elutropic series.

The different polarity of solvents of different structure is clearly perceptible both in the interactions with one another and in the interactions with other molecules.

The dipole moment of a substance determines the solubility thereof or ability thereof to act as a solvent itself. The general rule here is that like dissolves like. Thus, polar substances have good solubility in polar solvents, but sparing solubility in nonpolar solvents. In contrast, nonpolar substances have good solubility in nonpolar solvents, such as benzine or cyclohexane, but sparing solubility in polar solvents.

Furthermore, the dipole moment also correspondingly influences the boiling point of polar solvents.

Polar solvents in connection with the present invention are those solvents wherein a polar atomic bond is present in the molecules. For example, polar solvents in the context of the present invention are ketones, such as acetone, ethers, such as tetrahydrofuran, diethyl ether, esters, such as ethyl acetate, and the like.

If the polar solvents do not have an X—H bond as defined above, where X is not C (carbon), the solvents cannot release any protons by dissociation.

Such substances are referred to as aprotic polar solvents. They have sparing miscibility with nonpolar solvents, and improved solubility of and in polar substances. Examples of aprotic polar solvents in the context of the present invention are ketones, such as acetone, ethers, such as diethyl ether or tetrahydrofuran, esters, such as ethyl acetate, lactones, such as 4-butyrolactone, nitriles, such as acetonitrile, tertiary carboxamides, such as N,N-dimethylformamide, urea derivatives, such as tetramethylurea or dimethylpropyleneurea (DMPU), sulfoxides, such as dimethyl sulfoxide (DMSO), or sulfones, such as sulfolane.

The term “mixture” is used here as known to those skilled in the art and relates to the combination of substances or substance streams so as to achieve a very homogeneous composition (homogeneity). A mixture comprises at least two mutually miscible constituents. The term “miscible” is understood to mean the ability of substances to form homogeneous mixtures with one another in any ratio.

The above-defined term “solution” in the context of the present invention should be clearly distinguished from the term “mixture”. In contrast to the solution, for example, individual molecules of a solvent are not present surrounded by a solvate shell of the other solvent in the mixture. Instead, the solvents have similar polarities and/or comparable dipole moments, and so the solvents are capable of forming homogeneous mixtures with one another. In connection with the process according to the invention, the term preferably relates to a mixture of at least two different solvents.

The inventive coprecipitate can be produced, for example, as follows: the active pharmaceutical constituent of sparing solubility in aqueous solution and the pharmaceutically acceptable carrier are stirred in a mixture of an aprotic polar solvent and a protic solvent in a vessel, preferably at elevated temperature, for example approx. 30° C. For coprecipitation, further protic solvent is added while stirring continuously. After the coprecipitation, the product is filtered off, preferably using reduced pressure. Thereafter, the solid coprecipitate is washed repeatedly with the protic solvent.

The coprecipitate produced by the process according to the invention comprises a phosphodiesterase 5 inhibitor (PDE5 inhibitor) and a pharmaceutically acceptable carrier, wherein the phosphodiesterase 5 inhibitor (PDE5 inhibitor) is of sparing solubility in an aqueous solution.

Preferably, the phosphodiesterase 5 inhibitor (PDE5 inhibitor) in the coprecipitate produced by the process according to the invention is of sparing solubility particularly in water.

Likewise preferably, the phosphodiesterase 5 inhibitor (PDE5 inhibitor) in the coprecipitate produced by the process according to the invention is sildenafil, vardenafil or tadalafil, especially tadalafil.

Additionally preferably, the carrier in the coprecipitate produced by the process according to the invention is a copolymer consisting of 2 or more different acrylic acid derivatives of the general formula (I) according to the above definition of the general formula (I), and/or a cellulose acetate, a starch derivative or an oligosaccharide. The copolymer preferably consists of 2 or more, especially 2, 3, 4 or 5, in particular 3, different acrylic acid derivatives of the general formula (I) according to the definition of this formula given above in this connection. Additionally preferably, the cellulose acetate is cellulose diacetate, cellulose triacetate, an incomplete hydrolysate thereof; cellulose acetate phthalate, or cellulose acetate butyrate, especially cellulose acetate butyrate or cellulose acetate phthalate, the starch derivative is a crosslinked starch, an acetylated starch or a substituted n-octenylsuccinate of starch, and the oligosaccharide is a disaccharide such as maltose, lactose or sucrose. More particularly, the carrier in the coprecipitate produced by the process according to the invention is poly(butyl methacrylate, 2-dimethylaminoethyl methacrylate, methyl methacrylate).

In addition, the phosphodiesterase 5 inhibitor (PDE5 inhibitor) and the pharmaceutically acceptable carrier are preferably present in the coprecipitate produced by the process according to the invention in a weight ratio of 1:2 to 2:1. More particularly, the phosphodiesterase 5 inhibitor (PDE5 inhibitor) and the pharmaceutically acceptable carrier are present in the coprecipitate produced by the process according to the invention in a weight ratio of 1:1.

Likewise preferably, in the coprecipitate produced by the process according to the invention, the phosphodiesterase 5 inhibitor (PDE5 inhibitor) is enclosed by the pharmaceutically acceptable constituent.

Accordingly, in one embodiment of the process according to the invention for production of an inventive coprecipitate, the terms used are each as defined above in connection with the inventive coprecipitate.

Preferably, the aprotic polar solvent in the process according to the invention for production of the inventive coprecipitates is an ether; more particularly, this ether is tetrahydrofuran.

The aprotic polar solvents and protic solvents used in the process according to the invention are not restricted to pure solvents of their respective category. Both the aprotic polar solvent used in the process according to the invention and the protic solvent may comprise proportions of the solvent of the other category in each case. For example, tetrahydrofuran as the preferred aprotic polar solvent may comprise water as a protic solvent, in which case the lower concentration limit of the water content, however, is not below 1 ppm and the upper concentration limit of the water content is below 50%.

In addition, the solvents can be admixed with acids or bases, preferably acids, in which case the acid or base content should not exceed a concentration of 1 N. For example, a solvent contains approx. 1% HCl.

Accordingly, in one embodiment of the process according to the invention, the aprotic polar solvent is an ether, especially tetrahydrofuran, and/or the protic solvent is an alcohol or water, especially water.

In addition, dimethyl sulfoxide is also suitable as aprotic polar solvent in the process according the invention.

Preferably, in the process according the invention for producing the inventive precipitate, the phosphodiesterase 5 inhibitor (PDE5 inhibitor) is tadalafil and the pharmaceutically acceptable carrier poly(butyl methacrylate, 2-dimethylaminoethyl methacrylate, methyl methacrylate). In addition, tadalafil and poly(butyl methacrylate, 2-dimethylaminoethyl methacrylate, methyl methacrylate) are present in the process according to the invention in a weight ratio of 2:1 to 1:2, for example in a weight ratio of 2:1, 1:1 or 1:2, special preference being given to the weight ratio of 2:1 to 1:1, particularly to the weight ratio of 2:1 or 1:1.

The coprecipitation of the phosphodiesterase 5 inhibitor (PDE5 inhibitor) and of the pharmaceutically acceptable carrier can be increased by increasing the protic character of the solution. This can be accomplished in the simplest case by adding acid to the mixture in step b) of the process according to the invention and thus by an increase in the number of protons in the mixture. Since water is subject to what is called autoprotolysis and itself releases protons, it is also possible to add additional water to the mixture and hence to increase the protic character of the mixture.

Accordingly, the present invention also relates to an embodiment of the process according to the invention for producing the inventive coprecipitate, in which the protic character of the mixture is increased by adding additional protic solvent.

The inventive coprecipitate can be processed further directly or in another form as a medicament.

Accordingly, the present invention further provides a medicament comprising the above-defined coprecipitate.

The term “medicament”, also called pharmaceutical, is used here as known to those skilled in the art, and relates to substances and formulations of substances intended, for use on or in the human or animal body, in order to:
heal, alleviate, prevent or recognize disorders, conditions, physical injury or pathological complaints;
repel, eliminate or render harmless pathogens, parasites or substances foreign to the body;
recognize or influence the nature, the state or the functions of the body, or psychological states; and/or
replace active ingredients or body fluids generated by the human or animal body.

In addition, the term “medicament” also includes, for example, articles which comprise a medicament as defined above or to which such a medicament has been applied, and which are intended for lasting or temporary contact with the human or animal body.

The inventive coprecipitate can be used to treat a disorder in which inhibition of phosphodiesterase 5 is of therapeutic benefit. The phosphodiesterase 5 inhibitor (PDE5 inhibitor) is as defined above.

More particularly, the inventive coprecipitate is suitable for treatment of a disorder selected from the group consisting of erectile dysfunction, premature ejaculation, sexual dysfunction in women, polycystic ovary syndrome (PCOS), benign prostate hyperplasia (BPH), period pain (dysmenorrhea), cerebrovascular disease, stroke, optic neuropathy, osteoporosis, cachexia, hydropic heart decompensation, ischemic heart disease, peripheral arterial disease, hypertension, thrombocythemia, autoimmune disease, inflammation disease, cancer, a disease caused by gut motility disorders, hyperglycemia, glucose tolerance disorders, diabetes, insulin resistance syndrome, glomerular renal insufficiency, renal inflammation, renal failure, increased intraocular pressure, glaucoma, macular degeneration, respiratory disease, tubulointerstitial lung disease, acute respiratory distress syndrome (ARDS), pulmonary hypertension, urological disorders, overactive bladder, bladder outlet obstruction and incontinence.

PDE5 itself is a key enzyme in the regulation of the cGMP level in the smooth muscle of erectile corpus cavernosum tissue. The physiological mechanism of erection includes the release of nitrogen oxide in the corpus cavernosum during sexual stimulation. The nitrogen oxide released subsequently activates the enzyme guanylate cyclase, which leads to an increased level of cGMP, which in turn causes the relaxation of the smooth muscle in the corpus cavernosum. The relaxation of the smooth muscle enables the flow of blood into the corpus cavernosum and thus leads to erection.

The inhibition of PDE5 inhibits the degradation of cGMP. Therefore, it enables the inhibition of PDE5 to maintain the cGMP level, which consequently also leads to lasting relaxation of the smooth muscle of the corpus cavernosum. This enables (longer-)lasting and/or erection of the corpus cavernosum.

In order that the penis as the corpus cavernosum in question becomes erect at all, the following events must occur: (1) widening of those arteries which regulate blood flow to the cavities of the corpus cavernosum; (2) relaxation of the trabecular smooth muscle, which facilitates the “occlusion” of the penis with blood; and (3) compression of the veins by the expanding trabecular walls in order to prevent the venous outflow of the blood.

The inability to maintain an erection or to maintain it for a sufficiently long period is referred to colloquially as impotence. In a neutral sense, this term is nowadays covered by the term “erectile dysfunction”.

Possible causes which have been identified for impotence are, as well as neurogenic, endocrinological and psychological reasons, also vasculogenic reasons, and the latter are considered to be the most common reason for impotence. Vasculogenic impotence is caused by changes in blood flow into and out of the penis.

For treatment of such erectile dysfunction, PDE5 inhibitors have been found to be extremely effective. As well as sildenafil and vardenafil, tadalafil in particular has turned out to be a particularly suitable PDE5 inhibitor.

Consequently, the above-defined coprecipitate comprising an active pharmaceutical constituent and a pharmaceutically acceptable carrier, wherein the active pharmaceutical constituent is sparingly soluble in an aqueous solution, is preferentially suitable for treatment of erectile dysfunction.

Pulmonary hypertension and pulmonary arterial hypertension are understood to mean disorders characterized by an increasing rise in vascular resistance and a rise in blood pressure in the pulmonary circulation, these symptoms often being associated with subsequent right-ventricular heart failure. The patients often suffer from severely weakened physical capacity, circulation disorders and tiredness.

Pulmonary hypertension often occurs as the consequence of chronic obstructive pulmonary disease (COPD), but secondary occurrence of pulmonary hypertension is also possible as a consequence of other disorders, for example pulmonary embolism, pulmonary fibrosis, sarcoidosis, asthma, AIDS, sickle cell anemia, sclerodermia and congenital heart defects.

In contrast to secondary pulmonary hypertension, primary (or idiopathic) pulmonary hypertension, which occurs rarely, is not a complication of another underlying disorder. Consequently, in the case of primary (or idiopathic) pulmonary hypertension, in contrast to secondary hypertension, the exact causes are not known.

Possible causes which are being discussed for increased blood vessel tone are enhanced release of factors which contract blood vessels, for example endothelin and thromboxane, and reduced production of relaxing factors, for example nitrogen monoxide and prostacyclin.

Lasting success in treatment of the symptoms of pulmonary hypertension requires that any underlying disorder leading to pulmonary hypertension is eliminated in a timely manner, specifically before any fixed pulmonary hypertension has occurred. If such a treatment is carried out too late or is medically impossible, the only option is palliative treatment with medicaments, or a lung or heart and lung transplant. For this reason, children with congenital heart defects are operated on as early as possible, such that pulmonary hypertension cannot develop. The technical means required for this purpose (heart-lung machine) and surgical experience in the correction of congenital heart defects, even in babies and infants, are available.

In general, medicament therapy of pulmonary hypertension is considered to be difficult. As of recently, however, some drugs are available for the treatment of pulmonary hypertension, which have also—in some cases with restrictions—been approved for treatment.

Cardiac disorders are classified by what is called the NYHA classification, a scheme originally published by the New York Heart Association for the classification of cardiac disorders. It is most commonly used for classification of heart failure into different, stages according to the physical capacity of the patient (NYHA stages I to IV); in addition, there are adaptations to other disorders, for example pulmonary hypertension.

According to severity, the International Guidelines of the Consensus Commission of the 3^{rd} PAH World Symposium in Venice in 2003 (Galie N. et al: Comparative analysis of clinical trials and evidence-based treatment algorithm in pulmonary arterial hypertension. J. Am. Coll. Cardiol. 2004 Jun. 16; 43(12 Suppl S): 81S-88S) at NYHA stage III (cardiac disease resulting in marked limitation of physical activity, ordinary physical activity results in symptoms of fatigue, palpitation, dyspnoea or angina), recommend, as well as endothelin receptor agonists or prostacyclin analogs, also PDE5 inhibitors which widen the blood vessels.

Consequently, the above-defined coprecipitate comprising an active pharmaceutical constituent and the pharmaceutically acceptable carrier, wherein the active pharmaceutical constituent is of sparing solubility in an aqueous solution, is also preferentially suitable for treatment of pulmonary hypertension.

The inventive coprecipitate can either be administered directly or processed further.

The present invention further provides a process for producing the inventive medicament, comprising the steps of:
a) comminuting the inventive coprecipitate, and
b) isolating comminuted coprecipitate particles with a maximum diameter of 500 μm.

The term “comminuting” is used here as known to those skilled in the art and relates to the mechanical displacement of the particle size distribution, for example of grains, to a finer size range. According to the grain size and hardness of the grain type, is between coarse crushing, fine crushing and rough grinding in the case of grain sizes of the starting material of 50 mm to 0.5 mm, and fine grinding, ultrafine grinding and colloid grinding in the case of grain sizes of 500 micrometers to below 5 micrometers. The naming of the products of the comminution ranges from chunks through lumps, chips, grit, flour, powder down to colloidal fineness. The equipment used includes jaw crushers, impact crushers, hammer mills, ball mills, colloid mills, material bed roll mills, single-shaft comminutors and many others.

The term “isolation” is used here as known to those skilled in the art and refers to the separation of substances on the basis of different substance properties, here preferably the particle size. Separation processes based on the size of particles are filtration, more specifically suction filtration., screening, sieving, sifting: plan sifting, wind sifting, membrane separation processes and reverse osmosis. In connection with the comminuted coprecipitate particles, isolation relates here more particularly to the operation of sieving, which is a mechanical separation process for size separation (classification) of bulk materials. This involves placing the material to be separated onto a sieve which is set in rotation or shaken.

A sieve is an apparatus for separation of solid substances according to the criterion of particle size, exploiting gravity as the driving force. As a result, at least two fractions are obtained, which differ in their minimum and maximum particle size respectively. The material applied is usually a solid mixture of different particle sizes (for example bulk material), but it may also be a solid mixture together with liquid, in connection with the process according to the invention for production of the inventive medicament preferably a solid mixture of different particle size.

The separation is effected through the sieve plate or sieve surface, which contains a multitude of orifices of equal size as the actual separation medium. This consists of metal (perforated sheet, wire mesh, metal grid or metal wires), plastic, rubber of varying hardnesses, or silk gauze. The size of the orifices is referred to as mesh size and defines the sieve cut. In most countries, the orifice is defined either in “mm” or in “μm”, but in the USA in “mesh” (number of meshes per inch, sometimes also number of orifices per square inch). Grains with a diameter greater than the mesh size remain on top (sieve oversize) and grain with a smaller diameter falls downward (sieve throughput). A grain of approximately equal size is called borderline grain. A sieve may consist of one or more sieve surfaces one on top of another, in Which case the sieve with the greatest mesh size in the sieve stack is at the top.

For the efficiency of a sieve, the cleanliness of the sieve surface is of great importance. Especially the blockage of the sieve orifices by borderline grain must be prevented by suitable measures (for example brushes, balls, chains, rubber cubes, which are included on or below the sieve).

In addition, it is known to those skilled in the art that, in industrial(scale) applications, sieve surfaces are agitated to particular movements by a drive to improve the sieve performance (sieving machine). The movement of the sieve surface serves to transport the material applied further in longitudinal direction, to throw the borderline grain out of the mesh orifices, and to sustain the separation (sieve efficiency).

The inventive coprecipitates and the coprecipitates isolated in the process for producing the inventive medicament, however, are only balls with a circular cross section in the ideal case. In general, however, the comminuted coprecipitate particles do not have a circular cross section. Therefore, the term “diameter” in the context of the present invention is also applied to only approximately spherical coprecipitates, which have, for example, an elliptical, sickle-shaped or semicircular cross section, or even an essentially rhombus-shaped, square or rectangular cross section, although the cross section in the case of a rhombus, square or rectangular shape has rounded corners.

The term “maximum diameter” in connection with the isolation of comminuted coprecipitate particles refers to the dimension of the diameter of the coprecipitates which can still pass through the meshes of the particular sieve.

In this regard, a special case is considered hereinafter, namely that of coprecipitates of elongated form whose diameter in longitudinal direction is greater than the mesh size of the sieve used. Such coprecipitates should he able to pass through the meshes of the sieve only when they are aligned over the sieve such that the longitudinal axis thereof with the greater diameter is aligned vertically over the sieve. If these coprecipitates, in contrast, come to rest on the sieve with the longitudinal axis parallel thereto, they should actually not be able to pass through the meshes of the sieve, since the diameter thereof in longitudinal direction is greater than the mesh size of the sieve.

In this regard, it is known to the person skilled in the art in connection with the aforementioned agitation of the sieve surface and the above definition of the term “diameter” that the agitation of the sieve surface can align the coprecipitate particles such that it can pass through the mesh with a diameter smaller than the mesh size of the sieve.

Consequently, the term “maximum diameter” relates to the maximum diameter relevant under the conditions described above for a particle which can still pass through the meshes of the sieve. The maximum diameter of the comminuted coprecipitate particles in the process according to the invention is preferably 500 μm.

The particle size can be measured, for example, with the aid of laser diffraction. In the determination of the particle size with the aid of laser diffraction, it is possible, for example, to use an instrument of the Mastersizer 2000 type.

Laser diffraction as a method for determination of particle size is based on the effect that particles which pass through a laser beam scatter light at an angle directly correlated to the particle size thereof. In this context, it is observed that the scatter angle measured increases logarithmically with decreasing particle size. Consequently, large particles scatter the light with high intensity at relatively small angles; small particles, in contrast, scatter the light at wide angles, but with low intensity.

Instruments based on the principle of laser diffraction utilize this behavior to determine particle sizes. A typical instrument for this consists of a laser which generates coherent light of a particular wavelength, of a series of detectors which measure the light pattern generated over a broad spectrum of angles, and of a kind of sample presentation system in order to ensure that the material tested is conveyed through the laser beam as a homogeneous particle stream in a defined, reproducible state of dispersion.

Modern instruments are also equipped with modules for the analysis of liquid dispersions, and also for the analysis of both wet and dry aerosols.

In order to calculate the distribution of the particle sizes in laser diffraction, the scatter pattern of a sample is compared with an appropriate optical model. Typically, two different models are used here, namely the Fraunhofer approximation and the Mie theory, but these will not be discussed any further in the present matter.

The dynamic measurement range is directly correlated to the angle range of the scatter angle measurement. Modern instruments measure from about 0.02 degree up to more than 140 degrees. The wavelength of the light used for the measurements is likewise important. Small wavelengths (e.g. blue light sources) exhibit higher sensitivity compared to particles in the submicron range.

As long as the particle diameter is large compared to the wavelength of the laser used (particles of >10 μm diameter), laser diffraction is the only significant phenomenon observed.

If the particle diameters, however, are in the same order of magnitude as the wavelengths of the laser used, particle-wave duality of electromagnetic radiation becomes relevant. In such cases, a more complex theory is applied to the diffraction, in which all interactions between light and particles are taken into account.

In the process according to the invention, the preferred maximum diameter of the comminuted coprecipitate particles, at 500 μm, is well above the critical diameter of 10 μm for the measurement range of laser diffraction. Therefore, laser diffraction is suitable without restriction for determination of the maximum particle diameter mentioned for the comminuted coprecipitate particles.

The process according to the invention for producing the inventive medicament preferably additionally comprises the step of
c) mixing
   i) the coprecipitate obtained in step a) or b),
   ii) a hinder and/or tablet disintegrant such as cellulose, a cellulose derivative, an oligo- or polysaccharide,
   iii) optionally an emulsifier, especially sodium laurylsulfate, and
   iv) optionally a lubricant, especially magnesium stearate, and
d) optionally pressing the mixture as obtained in step c) to a tablet.

Optionally, in the abovementioned additional step c) of the process according to the invention for producing the inventive medicament, the coprecipitate obtained in step a) or b) can be mixed with a binder and/or filler and/or tablet disintegrant.

The term “binder” is used here as known to those skilled in the art and relates to those compounds which improve adhesion. Binders include, but are not restricted to, water, ethanol, polyvinylpyrrolidone, starch, gelatin or sugars, including sucrose, dextrose, molasses and lactose, and microcrystalline cellulose.

The terms “cellulose”, “cellulose derivative”, “oligo- and polysaccharide” are used here as defined above.

Optionally, in the abovementioned additional step c) of the process according to the invention for producing the inventive medicament, the coprecipitate obtained in step a) or b) can be mixed with a binder and/or tablet disintegrant.

The term “tablet disintegrant” is used here as known to those skilled in the art and relates to auxiliaries which ensure the rapid decomposition of tablets in water or gastric juice, and hence the release of the active ingredient in absorbable form. According to the mechanism of action, the substances in connection with the present invention are those which increase the porosity of the compressed articles and have a high absorption capacity for water, for example starch, cellulose derivatives, alginates, dextrans, crosslinked polyvinylpyrrolidone or hydrophilizing agents which ensure the vetting of the compressed particles, for example polysorbates (e.g. Tween® 20, Tween® 21, Tween® 40, Tween® 60, Tween® 61, Tween® 65, Tween® 80, Tween® 81 and Tween® from ICI America, Inc.). Tablet disintegrants include the following compounds, but are not restricted thereto: crosslinked polyvinylpyrrolidones (e.g. crospovidone, for example Polyplasdone® XL obtainable from GAF), crosslinked carboxymethylcellulose (e.g. croscarmellose, for example Ac-di-sol® from FMC); alginic acid, calcium silicate and sodium carboxymethyl starches (e.g. Explotab®, obtainable from Edward Medell Co., Inc.); methylcellulose; agar bentonite; alginic acid; calcium carbonate, polysorbate; sodium laurylsulfate; or lactose and lactose derivatives, such as agglomerated lactose, for example Tablettose® 80.

Preferred binders or tablet disintegrants are cellulose, cellulose derivatives, oligo- or polysaccharides.

In addition, an emulsifier may be present in step c).

The term “emulsifier” is used here as known to those skilled in the art and relates to substances which enable or facilitate the homogeneous distribution of two or more immiscible phases, and at the same time prevent the separation of the phases. Emulsifiers are divided into two main groups, into those which have usually colloidal solubility either in the oil phase or in the water phase, in some cases also in certain oil and water phases at the same time, and those which are soluble neither in the oil phase nor in the water phase. The latter pulverulent emulsifiers, however, are only of minor significance. The former group comprises surface-active substances; they may at the same time be wetting agents. This group is divided into anion-active or anionic, cation-active or cationic, nonionogenic or nonionic, and ampholytic emulsifiers. The anion-active emulsifiers include the alkali metal salts of the fatty acids, i.e. the soaps (e.g. ammonium stearate, palmitate, oleate or linoleate, potassium stearate, palmitate, oleate or linoleate, sodium stearate, palmitate, oleate or linoleate, etc.), the alkaline earth metal or heavy metal salts of higher fatty acids, also called metal soaps (e.g. calcium palmitate or stearate, zinc palmitate or stearate, magnesium palmitate or stearate, aluminum palmitate or stearate, zinc myristate, calcium oleate, etc.), organic soaps (e.g. mono-, di- or triethanolamine oleate, mono-, di- or triethanolamine stearate, etc., diethylethanolamine stearate, 2-amino-2-methyl-1-propanol stearate, morpholine stearate etc.), sulfated compounds (e.g. sodium laurylsulfate, sodium cetyl sulfate, triethanolamine laurylsulfate, sodium monolaurylglycerylsulfate, turkey red oil etc.), sulfonated compounds (e.g. sodium cetylsulfonate, Igepon T, Aerosol OT etc.), phosphorylated compounds (e.g. sodium laurylphosphate), lamepons, bile acid salts (e.g. sodium glycocholate), saponins etc. A preferred emulsifier in the context of the process according to the invention is sodium laurylsulfate,

In addition, a lubricant may be present in step c).

Lubricants are those auxiliaries which improve the flow properties of the coprecipitate intended for tableting in the filling funnel and filling shoe of the tableting machine. Lubricants include, but are not restricted to, stearic acid, polyethylene glycol or stearates, for example magnesium stearate. A preferred emulsifier in the context of the process according to the invention is magnesium stearate.

Optionally, the mixture obtained in step c) can be pressed to a tablet in step d).

The term “pressing” is used here as to the person skilled in the art and relates to the mechanical operation in Which exertion of pressure via dies or corresponding molds, for example in what is called a tableting press, produces individually dosed solid drug forms, called tablets, from powders or granules. The shapes of the tablets produced by this operation may be different. Tablets to be taken orally are preferably of oblong-biconvex shape (round and curved on both sides).

The figures and examples which follow are presented in order to further illustrate the invention claimed. The scope of protection of the present invention shall not be restricted thereby.

### FIGURES

FIG. 1 shows release curves for the tablets from example 6, i.e. with a coprecipitate consisting of tadalafil and Eudragit in a ratio of 2:1, compared to the reference formulation, Cialis® 20 mg. The conditions for release of the tadalafil from the tablets are 1000 ml of 0.1 N HCl+0.5% SLS (sodium laurylsulfate), 37° C. and 50 rpm (revolutions per minute).

### LEGEND TO FIGURES

[symbol] tablet comprising tadalafil coprecipitate, stored for 4 weeks at 40° C./75% relative air humidity.

[symbol] tablet comprising tadalafil coprecipitate, stored for 4 weeks at 25° C./60% relative air humidity.

♦ reference is Cialis® 20 mg

Δ tablet comprising tadalafil coprecipitate (without stable storage, i.e. freshly produced)

### EXAMPLES

### Example 1

### Production of Coprecipitates of Tadalafil and HPMCP

2.5 of tadalafil and 2.5 g of HPMCP (hydroxypropylmethylcellulose phthalate) HP-55 are dissolved in a mixture of 17.0 g of tetrahydrofuran and 3.0 g of deionized water in a vessel while stirring. For coprecipitation, 30 g of a 1% HCl solution are added while stirring continuously. On completion of the coprecipitation operation, the product is filtered off with the aid of a vacuum pump. The solid coprecipitate is washed three times with 33.0 g of water. After the washing operations, the product is dried in an oven at 50° C. for 20 hours. Thereafter, the dried product is crushed with a mortar and dried in an oven for a further 4 hours. Tablets can be pressed from the resulting product.

### Example 2

### Production of Coprecipitates of Tadalafil and Eudragit (1:1)

2.5 of tadalafil and 2.5 of Eudragit E100 are dissolved in a mixture of 17.0 g of tetrahydrofuran and 3.0 g of deionized water in a vessel while stirring and heating briefly to up to 30° C. For coprecipitation, 30.0 g of water are added while stirring continuously. On completion of the coprecipitation operation, the product is filtered of with the aid of a vacuum pump. The solid coprecipitate is then washed three times with 33.0 g of water each time. After the washing operations, the product is dried in an oven at 50° C. for 20 hours. Thereafter, it is crushed with a mortar and dried in an oven for a further four hours.

### Example 3

### Production of Coprecipitates of Tadalafil and Eudragit (2:1)

5.0 g of tadalafil and 2.5 g of Eudragit E100 are dissolved in a mixture of 34.0 g of tetrahydrofuran and 6.0 g of water in a vessel while stirring and heating briefly to up to 30° C. For coprecipitation, 60.0 g of deionized water are added while stirring continuously. On completion of the coprecipitation operation, the product is filtered off with the aid of a vacuum pump. The solid coprecipitate is then washed three times with 60.0 g of water each time. After the washing operations, the product is dried in an oven at 50° C. for 20 hours. Thereafter, it is crushed with a mortar and dried in an oven for a further 14 hours.

### Example 4

### Production of Tablets from the Coprecipitate from Example 1

The coprecipitate from example 1 is crushed with a mortar and pestle and sieved through a sieve of pore size 500 μm. Thereafter, 40.8 g of the sieved coprecipitate are mixed with 59.7 g of microcrystalline cellulose (Avicel® PH 102), 225.0 g of agglomerated lactose (Tablettose® 80, from Meggle) and 23.0 g of crosslinked carboxymethylcellulose (Ac-Di-Sol®, lederle Labs.) and 1.0 g of sodium laurylsulfate at 23 rpm (rounds per minute) in a Turbula mixing machine for 10 minutes. 0.9 g of magnesium stearate is added to this mixture and the resulting mixture is mixed for 5 minutes. Tablets are pressed using a EK0 single punch tablet machine.

### Example 5

### Production of Tablets from the Coprecipitate from Example 2

The coprecipitate from example 2 is crushed with a mortar and pestle and sieved through a sieve of pore size 500 μm. Thereafter, 40.4 g of the sieved coprecipitate are mixed with 59.7 g of microcrystalline cellulose (Avicel® PH 102), 225.0 g of agglomerated lactose (Tablettose® 80, from Meggle) and 23.0 g of crosslinked carboxymethylcellulose (Ac-Di-Sol®, Lederle Labs.) and 1.0 g of sodium laurylsulfate at 23 rpm (rounds per minute) in a Turbula mixing machine for 10 minutes. 0.9 g of magnesium stearate is added to this mixture and the resulting mixture is mixed for 5 minutes. Tablets are pressed using a EK0 single punch tablet machine.

### Example 6

### Production of Tablets from the Coprecipitate from Example 3

The coprecipitate from example 3 is crushed with a mortar and pestle and sieved through a sieve of pore size 500 μm. Thereafter, 30.0 g of the sieved coprecipitate are mixed with 59.7 g of microcrystalline cellulose (Avicel® PH 102), 224.6 g of agglomerated lactose (Tablettose® 80, from Meggle) and 23.0 g of crosslinked carboxymethyl cellulose (Ac-Di-Sol®, Lederle Labs.) and 1.0 g of sodium laurylsulfate at 23 rpm (rounds per minute) in a Turbula mixing machine for 10 minutes. 0.9 g of magnesium stearate is added to this mixture and the resulting mixture is mixed for 5 minutes. Tablets are pressed using a EK0 single punch tablet machine.

The corresponding process par for production of the tablets are listed below:

| batch size: | 1000 tablets |
|---|---|
| decomposition time: | 30-45 seconds |
| rectangular tablet punch: | 12.5 * 6.5 mm, radius 4.6 mm |
| crushing strength: | ~80 kN |
| height: | 5.3 mm |
| length: | 12.4 mm |
| diameter: | 6.5 mm |
| primary pressing force: | ~7 kN |
| machine speed: | 3 |

The tablets of the tadalafil coprecipitate are stored in mono blisters (blister packs) made of PVC at 40° C./75% relative air humidity, 30° C./65% relative air humidity and 25° C./60% relative air humidity for a period of 4 weeks. The samples stored in mono PVC blisters at 40° C./75% relative air humidity are examined for stability after 4 weeks; the samples do not have any significant decomposition.

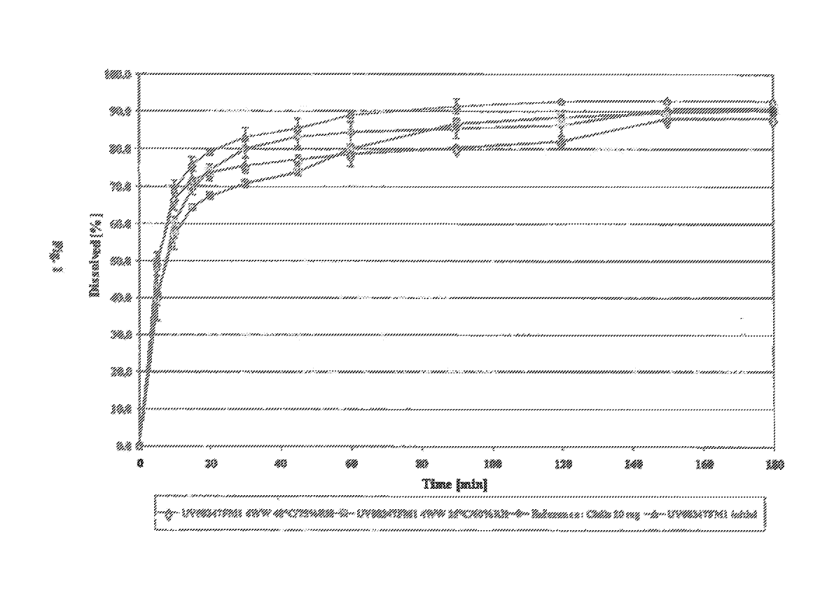

As is evident from FIG. 1, the release profiles of the coprecipitate-containing tablets measured in 0.1 N HCl, before and after storage for 4 weeks, had equally good release—with much simpler production—as the original Cialis® tablet. The release and hence also the bioavailability of tadalafil from the tablets comprising the inventive coprecipitates is much faster compared to the coprecipitates known from the prior art. For instance, a tablet comprising a coprecipitate containing tadalafil and Eudragit in a ratio of 1:1 exhibits 70% release of the active ingredient after 60 minutes compared to the 50% release of the active ingredient from a tablet comprising a coprecipitate containing tadalafil and HPMCP (hydroxypropylmethylcellulose phthalate) in a ratio of 1:1 (data not shown).

1. A coprecipitate comprising a phosphodiesterase 5 inhibitor (PDE5 inhibitor) and at least one pharmaceutically acceptable carrier, wherein the pharmaceutically acceptable carrier is a copolymer consisting of 2 or more different acrylic acid derivatives of the general formula (I) 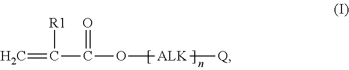
where, in any of the 2 or more different acrylic acid derivatives, each independently,
R1 is H or a straight-chain or branched C1-C6 alkyl radical,
n is 0 or 1,
ALK is a straight-chain or branched C1-C6 alkylene radical,
Q is H or —OR2, —NR2R3 or —N⁺R2R3R4, where R2, R3 and R4 are each independently a straight-chain or branched C1-C6 alkyl radical,
and/or the pharmaceutically acceptable carrier is a cellulose acetate, a starch derivative or an oligosaccharide.

2. The coprecipitate as claimed in claim 1, wherein the copolymer consists of 2 or more different acrylic acid derivatives of the general formula (I), where, in each of the 2 or more different acrylic acid derivatives, each independently,
R1 is H or a straight-chain C1-C4 alkyl radical, methyl, ethyl, propyl or butyl, especially methyl,
n is 0 or 1, especially 1,
ALK is a straight-chain C1-C4 alkylene radical, methylene, ethylene, propylene or butylene, especially methylene, ethylene or butylene,
Q is H or —NR2R3 where R2 and R3 are each independently a straight-chain C1-C4 alkyl radical, methyl, ethyl, propyl or butyl, especially methyl;
the cellulose acetate is cellulose diacetate, cellulose triacetate, an incomplete hydrolysate thereof, cellulose acetate phthalate, or cellulose acetate butyrate, especially cellulose acetate phthalate or cellulose acetate butyrate,
the starch derivative is a crosslinked starch, an acetylated starch or a substituted n-octenylsuccinate of starch, and
the oligosaccharide is a disaccharide such as maltose, lactose or sucrose.

3. The coprecipitate as claimed in claim 1, wherein the copolymer is poly[butyl methacrylate, 2-dimethylaminoethyl methacrylate, methyl methacrylate].

4. The coprecipitate as claimed in claim 1, wherein the phosphodiesterase 5 inhibitor (PDE5 inhibitor) is sildenafil, vardenafil or tadalafil.

5. The coprecipitate as claimed in claim 1, wherein the phosphodiesterase 5 inhibitor (PDE5 inhibitor) and the pharmaceutically acceptable carrier are present in a weight ratio of 1:2 to 2:1.

6. The coprecipitate as claimed in claim 1, wherein the phosphodiesterase 5 inhibitor (PDE5 inhibitor) is enclosed by the pharmaceutically acceptable carrier.

7. A method for producing a coprecipitate according to claim 1, comprising the steps of:
dissolving the phosphodiesterase 5 inhibitor (PDE5 inhibitor) and the pharmaceutically acceptable carrier in a mixture of an aprotic polar solvent and a protic solvent,
b) coprecipitating the phosphodiesterase 5 inhibitor (PDE5 inhibitor) and the pharmaceutically acceptable carrier by increasing the protic character of the mixture of the solvents, and
c) removing the coprecipitate from the mixture of the solvents.

8. The method as claimed in claim 7, wherein the phosphodiesterase 5 inhibitor (PDE5 inhibitor) and/or the pharmaceutically acceptable carrier is/are as defined in claim 2.

9. The process as claimed in claim 7, wherein the polar solvent is an ether, and/or wherein the protic solvent is an alcohol or water.

10. The method as claimed in claim 7, wherein the phosphodiesterase 5 inhibitor (PDE5 inhibitor) is tadalafil and the pharmaceutically acceptable carrier is poly[butyl methacrylate, 2-dimethylaminoethyl methacrylate, methyl methacrylate], tadalafil and poly[butyl methacrylate, 2-dimethylaminoethyl methacrylate, methyl methacrylate] being present in a weight ratio of 2:1 to 1:2.

11. The method as claimed in claim 7, wherein the polarity of the mixture is increased by adding additional protic solvent.

12. A medicament comprising the coprecipitate as claimed in claim 1.

13. A method for the treatment of a disorder in which the inhibition of phosphodiesterase 5 is of therapeutic benefit, the method comprising administering to a subject the coprecipitate of claim 1.

14. A method for producing a medicament as claimed in claim 12, comprising the steps of:
a) comminuting the coprecipitate as claimed in claim 1, and
b) isolating comminuted coprecipitate particles having a maximum diameter of 500 μm.

15. The method as claimed in claim 14, additionally comprising the steps of:
c) mixing
i) the coprecipitate obtained in step a) or b),
ii) a filler/binder and/or tablet disintegrant such as cellulose, a cellulose derivative, an oligo- or polysaccharide,
iii) optionally an emulsifier, especially sodium laurylsulfate, and
iv) optionally a lubricant, especially magnesium stearate, and
d) optionally pressing the mixture as obtained in step c) to a tablet.

16. The coprecipitate as claimed in claim 1, wherein the copolymer consists of 3, 4 or 5 different acrylic acid derivatives of the general formula (I),
where, in each of the 3, 4 or 5 different acrylic acid derivatives, each independently,
R1 is H or a straight-chain C1-C4 alkyl radical, methyl, ethyl, propyl or butyl, especially methyl,
n is 0 or 1, especially 1,
ALK is a straight-chain C1-C4 alkylene radical, methylene, ethylene, propylene or butylene, especially methylene, ethylene or butylene,
Q is H or —NR2R3 where R2 and R3 are each independently a straight-chain C1-C4 alkyl radical, methyl, ethyl, propyl or butyl, especially methyl;
the cellulose acetate is cellulose diacetate, cellulose triacetate, an incomplete hydrolysate thereof, cellulose acetate phthalate, or cellulose acetate butyrate, especially cellulose acetate phthalate or cellulose acetate butyrate,
the starch derivative is a crosslinked starch, an acetylated starch or a substituted n-octenylsuccinate of starch, and
the oligosaccharide is a disaccharide such as maltose, lactose or sucrose.

17. The coprecipitate as claimed in claim 1, wherein the phosphodiesterase 5 inhibitor (PDE5 inhibitor) is tadalafil.

18. The coprecipitate as claimed in claim 1, wherein the phosphodiesterase 5 inhibitor (PDE5 inhibitor) and the pharmaceutically acceptable carrier are present in a weight ratio of 1:1 or 2:1.

19. The method as claimed in claim 7, wherein the polar solvent is tetrahydrofuran, and/or wherein the protic solvent is water.

20. The method as claimed in claim 7, wherein the phosphodiesterase 5 inhibitor (PDE5 inhibitor) is tadalafil and the pharmaceutically acceptable carrier is poly[butyl methacrylate, 2-dimethylaminoethyl methacrylate, methyl methacrylate], tadalafil and poly[butyl methacrylate, 2-dimethylaminoethyl methacrylate, methyl methacrylate] being present in a weight ratio of 2:1 or 1:1.

21. A method for the treatment of a disorder selected from the group consisting of erectile dysfunction, premature ejaculation, sexual dysfunction in women, polycystic ovary syndrome (PCOS), benign prostate hyperplasia (BPH), period pain (dysmenorrhea), cerebrovascular disease, stroke, optic neuropathy, osteoporosis, cachexia, hydropic heart decompensation, ischemic heart disease, arteriosclerosis, peripheral arterial disease, hypertension, thrombocythemia, autoimmune disease, inflammation disease, cancer, a disease caused by gut motility disorders, hyperglycemia, glucose tolerance disorders, diabetes, insulin resistance syndrome, glomerular renal insufficiency, renal inflammation, renal failure, increased intraocular pressure, glaucoma, macular degeneration, respiratory disease, tubulointerstitial lung disease, a urological disease, overactive bladder, bladder outlet obstruction and incontinence, the method comprising administering to a subject the coprecipitate of claim 1.
